(19) United States
(12) Patent Application Publication
(10) Pub. No.: US 2021/0290078 A1
(43) Pub. Date: Sep. 23, 2021

(54) PRESSURE-SENSOR DEVICE FOR MEDICAL IN-VIVO APPLICATION

(71) Applicant: EMKA MEDICAL GMBH, Aschaffenburg (DE)

(72) Inventor: Felix GLOCKER, Aschaffenburg (DE)

(21) Appl. No.: 17/250,484

(22) PCT Filed: Jul. 29, 2019
(86) PCT No.: PCT/EP2019/000229

§ 371 Date: Jan. 27, 2021

(30) Foreign Application Priority Data
Jul. 27, 2018 (DE) 10 2018 005 911.2

(51) Int. Cl. A61B 5/0215; A61B 5/00

(52) U.S. Cl. CPC A61B 5/0215; A61B 5/6852; A61B 2562/0247; A61B 2562/168; A61B 2562/12

(57) ABSTRACT

A pressure sensor device for a medical in vivo application with at least one pressure transducer and an implantable probe, wherein the probe is proximally connected to the at least one pressure transducer, and the probe also comprises a catheter section and a measuring tip on the distal end of the probe, the probe having a longitudinal extension along the catheter section, wherein the catheter section comprises at least one lumen for producing a fluid connection from the measuring tip to the pressure transducer, and the lumen is filled with a transmission liquid, wherein the measuring tip comprises a tubular section with at least one laterally arranged opening, the at least one opening being covered with an elastic membrane, and wherein the filling quantity of the transmission liquid is determined such that, at a pre-determined temperature of the transmission liquid, the curvature of the membrane transverse to the longitudinal extension is substantially aligned with the contour of the measuring tip transverse to the longitudinal extension. The invention also relates to a measuring system and to a production method.

### FIELD

The invention relates to a pressure-sensor device for medical in-vivo application on a living human or animal body, to a measuring system and to a production process.

### BACKGROUND

The determination of blood pressure is one of the commonest and most widespread measures for registering vital parameters in medical diagnosis, therapy and care. The direct measurement of blood pressure is distinguished methodically from the indirect measurement of blood pressure.

In the case of direct blood-pressure measurement (IBP), which is also designated as invasive or bloody measurement, a sensing element is inserted directly into an artery via an arterial access. Alternatively, an access to an artery is put in and is connected to a measuring pickup outside the body. On account of the associated risks of infection and the requisite equipment infrastructure, the direct intra-arterial method is employed only in intensive therapy, on specially equipped hospital wards and in the operating room. In the case of indirect, or bloodless or non-invasive, blood-pressure measurement (NIBP), use is made of an electronic blood-pressure gauge or a blood-pressure cuff and a stethoscope. The values obtained with this method are somewhat less accurate than those obtained by direct blood-pressure measurement. Indirect blood-pressure measurement finds application far more frequently on wards and in doctors' surgeries and can also be carried out by the patient himself/herself outside of medical facilities.

For instance, from WO 2009/115223 a blood-pressure measuring system measuring directly outside the body is known having a system element that is known as a “pressure dome”, due to the domed design of the measuring chamber. A measured-value pickup to which such a dome is usually connected for the purpose of measuring or monitoring the pressure is known by the designation “transducer”, by which a measurement transformer in a suitable housing is understood which converts the pressures and changes in pressure usually transmitted via the membrane of the pressure dome into an electrical signal.

The membrane in such a dome serves for the infection-proof termination of the bloody connection to the patient. The transducer has been connected to an electronic diagnostic and monitoring instrument for displaying or evaluating the measurement signals. The advantage of such an arrangement is the possibility of designing the system element as an inexpensive one-way part that is easily disposed of, and thereby ensuring a hygienically impeccable and safe termination of the bloody fluid system; see WO 99/37983 A2.

Extracorporeal systems have the advantage that they can be easily calibrated via a three-way tap integrated into the line system conducted outside the body by opening of the side arm of the three-way tap at ambient pressure. In addition, on account of the room temperature which is typically constant in intensive-care rooms or operating rooms, further measures—for temperature compensation, for example—are not required.

In contrast, implanted pressure-measuring pickups have the disadvantage that they are exposed to fluctuations in temperature and cannot be zeroed by connection to the environment—that is to say, the zero-point drift of the implanted pressure transducer has to be very close to zero—that is to say, it has to remain within the measurement tolerances according to IEC 60601-2-34, even over a long period of months.

Furthermore, implantable pressure-measuring pickups are subject to severe restrictions with regard to the available installation space. For use even in large blood vessels, as a rule the transverse dimension of the pressure-measuring pickup must not amount to more than about 1 mm, because the invasive pressure transducer must not constitute an obstacle in the flow of blood. Therefore, spatially larger structural designs for the compensations of the temperature drift cannot be realized. Furthermore, the actual pressure-transducer circuit must include an electrical insulation barrier, both in the case of a capacitive pressure-measuring pickup with at least one movable capacitor plate and in the case of a piezoresistive pressure-measuring pickup with a silicon membrane with Wheatstone bridge etched on, further restricting the available installation space.

From DE 10 2015 116 648 A1 an implantable pressure-sensor appliance with a housing arrangement is known, in the case of which the housing arrangement exhibits outer walls and an inner volume, and the housing arrangement includes at least two pressure-transmitting membranes which each have a surface, and the surfaces are not arranged in one plane. In a reactive medium—such as in blood, for example—the proposed MEMS chips have to be protected. For this purpose, they are typically embedded in an incompressible and inert liquid and hermetically sealed in relation to the reactive medium in a housing. The liquid (for example, oil) serves as pressure-transmitting medium, so that the external pressure can be conducted to the MEMS chip via the housing. By way of housing material, titanium is proposed as being suitable, on account of its long-term stability and high biocompatibility.

Changes in temperature of several degrees Celsius can usually arise in the blood circulation of a patient, as a result of which changes occur in the volume of the pressure-transmitting medium within the housing. A further problem is the change in temperature during a sterilization—for instance, by means of ethylene oxide—in the course of which differences in temperature of about 30 degrees Celsius occur. The increases in volume and pressure resulting from this can damage the membranes or the MEMS chips in a conventional pressure-sensor housing. Since the housing usually has a low pliability or elasticity, said changes in the volume of the pressure-transmitting medium lead to great fluctuations in pressure within the housing. This factor is due, on the one hand, to the material properties of the housing and, on the other hand, to the housing walls which are relatively thick in relation to the size of the housing. As a result, the measured pressure values of the MEMS chip are falsified, since the pressure values to be measured have temperature-induced fluctuations in pressure in the interior of the housing superimposed on them.

For a MEMS-chip sensor arrangement, in U.S. Pat. No. 8,573,062 B2 the use is described of a pressure-transmitting membrane which covers a window that has been recessed into the side wall of the housing of the sensor arrangement. A pressure-transmitting membrane arranged on the end face of the housing is described in U.S. Pat. No. 8,142,362 B2.

In order to provide a pressure-sensor appliance that is predominantly insensitive to changes in temperature in the operating atmosphere and to the material stresses arising thereby, in DE 10 2015 116 648 A1 a housing arrangement is proposed that is to exhibit at least two pressure-transmitting membranes which each have a surface, the surfaces not being arranged in one plane. With the use of two pressure-transmitting membranes, two non-contiguous surfaces with greater pliability or elasticity are created for the housing of the implantable pressure-sensor appliance, so that the pressure in the interior of the housing is sufficiently reduced in the event of fluctuations in temperature. By the two pressure-transmitting membranes not being arranged in one plane, the stresses and forces on the housing material arising by virtue of a change in volume in the interior of the housing can also compensate one another at least partially, as a result of which the housing arrangement is said to gain in robustness.

With a view to solving the problem of excessive dimensions in the case of a pressure-measuring pickup with integrated temperature compensation, in US 2004/0073122 A1 an elongated arrangement is proposed that has been sealed on its distal end face with an elastic gel plug for the purpose of transmitting pressure. The gel plug is to be capable of being removed and exchanged by a user, and to be produced with a precursor and a plasticizer from a fully crosslinked multi-component silicone. For some embodiments it is proposed to cover the gel plug on the outside with an additional membrane consisting of a biocompatible material.

With a view to minimizing the drift, in US 2011/0209553 A1 it is proposed to equip a piezoresistive pressure-measuring pickup with two measuring chambers, one of the measuring chambers being hermetically sealed, and a predetermined reference pressure being intended to be set in the measuring chamber. The drift is to be minimized by comparative measurement. Jiachou Wang and Xinxin Li adopt a similar approach in “A dual-unit pressure sensor for on-chip self-compensation of zero-point temperature drift”, Journal of Micromechanics and Microengineering, 24 (2014) 085010 (DOI: 10.1088/0960-1317/24/8/085010).

In US 2011/0040206 A1 an arrangement with two membranes is proposed, in which one of the membranes is to be electrically deformable for the purpose of offset compensation.

An electrode arrangement for electrical stimulation of the right ventricle of the heart is known from DE 1919246 B2, with a connecting line consisting of a strand of insulating material and extending to a cardiac pacemaker, through which an electrical conductor leads to the electrode arranged on the outside of the strand of insulating material. The strand of insulating material is said to be so flexible that its insertion is to be possible solely by virtue of the entrainment by the bloodstream of the heart. This is said to have the disadvantage that electrical conductors that have been embedded into the strand of synthetic material so as to be non-displaceable in the longitudinal direction are subjected to severe strain in the event of bending of the strand of insulating material, so that they tear. This is said to occur particularly easily if the electrode arrangement is retracted a little, in order to change its position. In order to prevent the danger of tearing of the conductors in such an electrode arrangement, it is proposed to cause a tension-resistant core to extend through the strand of insulating material. In the case where use is made of two electrical conductors which, with a view to avoiding parasitic capacitances, are to extend, not closely adjacent, within a central duct in the case of a tubular design of the strand, an arrangement of the core centrally between the conductors is required for maximum protective action. The core is to accommodate the tensile forces arising in the strand of insulating material. By virtue of the arrangement between the electrical conductors, a varying bending capacity of the strand of insulating material in various bending directions is to be obtained.

An appliance for measuring body pressures or physiological pressures is known from DE 689 23 703 T2 and EP 0 417 171 B1, which device is said to be particularly useful for an incessant measurement of pressures. For this purpose, a pressure-transmitter catheter device for transmitting the physiological pressure to a pressure-transducer device is proposed, said pressure-transmitter catheter device exhibiting a hollow, flexible hose, with a first end for arrangement at a place at which the physiological pressure is to be measured and with a second end which is in communication with the pressure-transducer device, and a liquid which fills the hose and constitutes a connection to the pressure-transducer device, wherein a stopper has been positioned at the first end in the hose, wherein the stopper exhibits a material that is capable of transmitting pressure to the liquid which, in turn, transmits this pressure to the pressure-transducer device.

### SUMMARY

The object underlying the invention is therefore to provide a pressure-sensor device for medical in-vivo application that is suitable for simple and safe application on a living human or animal body and can be produced inexpensively.

In accordance with the invention, this object is achieved by a pressure-sensor device of the aforementioned type, with at least one pressure-measurement transformer and with an implantable probe, the probe being connected proximally to the at least one pressure-measurement transformer, the probe further including a catheter portion and a measuring tip at the distal end of the probe, the probe having a longitudinal extent along the catheter portion, the catheter portion exhibiting at least one lumen for establishing a fluid connection from the measuring tip to the pressure-measurement transformer, and the lumen being filled with a transmission liquid, the measuring tip including a tubular portion with at least one laterally arranged opening, the at least one opening being covered by an elastic membrane, and the filling quantity of the transmission liquid having been determined in such a way that at a predetermined temperature of the transmission liquid the curvature of the membrane at right angles to the longitudinal extent is substantially in alignment with the contour of the measuring tip at right angles to the longitudinal extent.

The object is achieved, furthermore, by a method for producing a pressure-sensor device for a medical in-vivo application with an implantable probe, the probe including a catheter portion and a measuring tip at the distal end of the probe, the probe having a longitudinal extent along the catheter portion, the catheter portion exhibiting at least one lumen for receiving a transmission liquid, the measuring tip including a tubular portion with at least one laterally arranged opening, the at least one opening being covered by an elastic membrane, with the following steps: controlling the temperature of the probe and of the transmission liquid to a predetermined temperature above the operating temperature specified for the pressure-sensor device, preferentially about 1 K to about 5 K higher than the specified operating temperature, in particular to about 40° C. to 46° C., in particular around 45° C., filling the lumen and the measuring tip with the temperature-controlled transmission liquid so far that the curvature of the membrane at right angles to the longitudinal extent is substantially in alignment with the contour of the measuring tip at right angles to the longitudinal extent, in particular does not protrude beyond the contour of the measuring tip at right angles to the longitudinal extent, and bubble-free sealing of the lumen.

With such an arrangement and such a method, it is possible for a pressure-sensor device according to the invention to be adjusted in such a way that the membrane is as free from stress as possible at the specified operating temperature during the measurement and hence an influence on the transmission of pressure to the pressure-measurement transformer is minimally slight. In addition, it is possible to implant a probe of a pressure-sensor device according to the invention without risk of damage by an only slightly larger sluice—for example, with an inner diameter merely 1F larger in comparison with the probe.

By virtue of the arrangement according to the invention, with the implantable probe it is also possible to monitor the blood pressure and, in particular, the pressure profiles of a patient, without electronic components such as pressure-measurement transformers likewise having to be implanted. As a result, the bureaucratic effort in connection with production and application of a pressure-sensor device according to the invention decreases considerably in comparison with known implantable pressure-measuring devices. Moreover, with the pressure-sensor device according to the invention it is also possible to monitor the pressure and the pressure profiles in a patient while he/she is being treated with instruments that cause severe electrical disturbances—for example, with an ablation catheter.

For a reliably tight fastening of the membrane to the probe, it is particularly favorable if the tubular portion of the measuring tip is encased by an elastic hose and the membrane consists of a material similar to that of the hose, both having preferentially been produced from a polyurethane. For a simple assembly, the elastic hose expediently exhibits an opening which overlaps the opening in the tubular portion, the membrane being tightly connected to the hose, the opening in the hose preferentially being slightly larger than the opening in the tubular portion.

The accuracy of measurement and, in particular, the usable resolution for the analysis of pressure signals can be improved if the tubular portion has been designed to be stable under pressure—for example, has been formed from a metal tube, preferentially from a medical stainless steel or a titanium alloy.

Furthermore, it is particularly advantageous if a flexible core has been embedded in the lumen which contains the transmission liquid. As a result, the danger of a kinking of the catheter portion, which might lead to a squeezing of the lumen and hence to a failure of the transmission of pressure to the at least one pressure-measurement transformer, is reduced. By virtue of the core in the lumen, the quantity of transmission liquid is reduced, which lessens not only the production costs for a pressure-sensor device according to the invention but also the influence of thermal expansion of the transmission liquid on the accuracy of measurement.

Even if a kinking of the probe were to occur in cramped spatial conditions such as may arise, for example, in an ambulance, the fluid passage in the lumen is not sealed completely, since the walls of the catheter portion are pushed apart by the kinked core. In this connection, it is particularly advantageous if the core consists of a polyamide, preferentially of a polyamide 11 or a polyamide 12. A core consisting of polyamide 12 (PA12) offers the particular advantage that a pressure-measuring device according to the invention can be employed effectively also in the course of surgical interventions under ongoing X-ray control, since in such an embodiment the core is resistant to X-radiation. Furthermore, a core consisting of polyamide 12 offers particular kink resistance of the catheter portion of the probe and hence a particularly high operational reliability of the pressure-measuring device according to the invention when employed under cramped spatial conditions, for example in an ambulance, in an air ambulance, in a mobile hospital or in a mobile medical facility. A core consisting of polyamide 11 (PA11) offers the merit of the applicability of common sterilizing procedures, is autoclavable, can be sterilized chemically with ethylene oxide, and by irradiation by means of gamma radiation. A core consisting of polyamide 11 also offers a high level of kink protection for the catheter portion of the probe. In addition, a core consisting of a polyamide 11 is to be classified as physiologically harmless.

For a good transmission of pressure to the transmission liquid via the membrane with slight falsifications as a result of stresses within the membrane, it is expedient if the at least one opening in the tubular portion has a larger dimension in the direction of longitudinal extent than in the circumferential direction; the ratio of the dimension in the direction of longitudinal extent to the dimension in the circumferential direction preferentially amounts to at least 5:1, in particular about 10:1.

A particularly broad operating range—within which the result of measurement is practically free from influence exerted by stresses within the membrane as a consequence of changes in the volume of the transmission liquid due to changes in temperature—can be obtained if the at least one opening in the tubular portion exhibits, in a direction at right angles to the longitudinal extent, a circumferential-arc portion covered by the membrane, which is substantially in alignment with the contour of the measuring tip at right angles to the longitudinal extent, and a chord portion which connects the opposing edges of the opening, the ratio of the length of the circumferential-arc portion to the length of the chord amounting to at least 1, preferentially between 1.33 and 1.67, more preferably between 1.5 and 1.6, particularly preferably about 1.57.

In tests, it has turned out to be particularly advantageous and operationally reliable if the transmission liquid comprises a water-insoluble perfluorinated liquid, the perfluorinated liquid having a boiling-point at normal pressure of at least 150° C., preferentially of about 165° C., and being completely evaporable. Such a transmission liquid practically does not react with other materials of the pressure-sensor device, does not outgas at the body temperatures occurring in vivo, and behaves inertly, even in the event of damage during handling, and hence minimizes the danger to nursing staff and patients.

Good pressure-transmitting behavior is obtained if the transmission liquid has a kinematic viscosity from about 2 mm²/s to 2.2 mm²/s at 25° C. and/or a coefficient of expansion of 0.0012 K⁻¹ and/or a surface tension of about 16 mN/m.

The invention can be realized economically particularly well with a measuring system containing at least one such pressure-sensor device.

### BRIEF DESCRIPTION OF THE DRAWINGS

The invention will be elucidated in more detail below on the basis of an embodiment example represented in the drawings. Shown are:

FIG. 1 is a schematic overall view of a pressure-sensor device according to the invention, partially in section;

FIG. 2 is an enlarged representation of a tubular portion of a measuring tip of the pressure-sensor device from FIG. 1;

FIG. 3 is the schematic overall view of a further pressure-sensor device according to the invention from FIG. 1, partially in section, with representation of the arrangement of a core;

FIG. 4 is a side view of the tubular portion from FIG. 2;

FIGS. 5 to 7 are enlarged cross-sectional views of the measuring tip of a pressure-sensor device according to the invention during varying stages of filling with a transmission liquid; and

FIGS. 8a to 8c are views of varying length ratios of circumferential-arc portion and chord portion on the basis of schematic cross-sectional views of the measuring tip.

### DETAILED DESCRIPTION

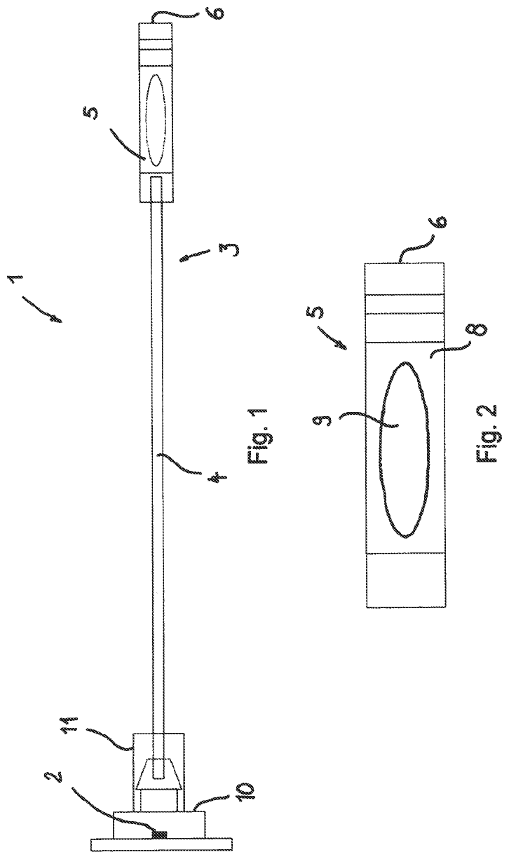

FIG. 1 shows a pressure-sensor device according to the invention, denoted overall by 1. The pressure-sensor device 1 includes at least one pressure-measurement transformer 2 and an implantable probe 3, the probe 3 being connected proximally to the at least one pressure-measurement transformer 2. The probe 3 comprises a catheter portion 4 and a measuring tip 5 at the distal end 6 of the probe 3. The probe 3 has a longitudinal extent along the catheter portion 4.

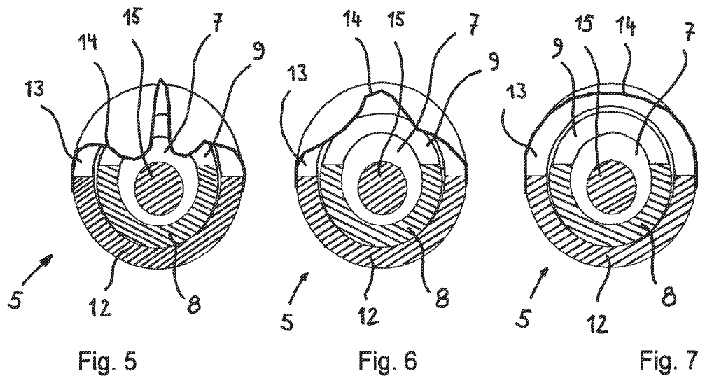

The catheter portion 4 exhibits at least one lumen 7, as can be discerned in FIGS. 5 to 7. The lumen 7 serves for establishing a fluid connection from the measuring tip 5 to the pressure-measurement transformer 2. For this purpose, the lumen 7 has been filled with a transmission liquid.

In tests, it has turned out to be particularly advantageous and operationally reliable to use by way of transmission liquid a water-insoluble perfluorinated liquid that has a boiling-point at normal pressure of at least 150° C., preferentially of about 165° C., and is completely evaporable. Such a transmission liquid practically does not react with other materials of the pressure-sensor device 1, does not outgas at the body temperatures occurring in vivo, and behaves inertly, even in the event of damage during handling, and hence minimizes the danger to nursing staff and patients. Such a transmission liquid is available with a kinematic viscosity from about 2 mm²/s to 2.2 mm²/s at 25° C., for example 2.1 mm²/s, and has a coefficient of expansion of about 0.0012 K⁻¹ and a surface tension of about 16 mN/m (±1 mN/m). In tests, a good pressure-transmission behavior has been achieved with such a transmission liquid.

The measuring tip 5 includes a tubular portion in the form of a thin metal tube 8, as shown in FIG. 2. The metal tube 8 has expediently been manufactured from a medical stainless steel or a titanium alloy and is dimensionally stable with respect to the pressures usually occurring in a human or animal body. The metal tube 8 exhibits a laterally arranged opening 9. The opening 9 has a larger dimension in the direction of longitudinal extent than in the circumferential direction. The preferentially oval opening 9 preferentially presents a ratio of the dimension in the direction of longitudinal extent to the dimension in the circumferential direction of at least 5:1, preferentially also more (6:1, 7:1, 8:1, 9:1, 10:1).

The catheter portion 4 has been tightly connected to a housing 10 of the pressure-measurement transformer 2 in a suitable manner known as such—for example, by a UV-curable adhesive. The junction between the catheter portion 4 and the housing 10 of the pressure-measurement transformer 2 has been expediently protected against damage with a supporting hose 11.

A TPE-A consisting of a polyether-block-amide block copolymer has proved itself by way of material for the catheter portion 4. The catheter portion 4 has been distally connected to the metal tube 8—for instance, glued in with a suitable adhesive.

The metal tube 8 has been covered at least radially with an elastic hose 12 consisting of a physiologically harmless elastic synthetic material—for example, consisting of a polyurethane. The hose 12 likewise exhibits an opening 13 which corresponds in position and shape to the opening 9 in the metal tube 8. For a simple assembly, it has been shown to be expedient if the opening 13 in the hose 12 is slightly larger than the opening 9 in the metal tube 8. As a result, the hose 12 can, for example, be simply glued at the edge of its opening 13 to the metal tube 8, so that the hose 12 has been secured against slipping.

The openings 13, 9 in the hose 12 and in the metal tube 8 have been covered by an elastic membrane 14. FIG. 4 shows schematically, in a type of radiograph, the positioning of metal tube 8, hose 12, membrane 14 and the openings 9 and 13 in relation to one another in a view from the side. The membrane 14 consists of a material that is significantly thinner than the hose 12 and that has been tightly sealed to the hose 12 hermetically by solvent bonding, as can be seen in the sectional views in FIGS. 5 to 7. By way of material for the membrane 14, likewise a polyurethane has been shown to be very suitable. The thickness of the membrane 14 should amount to no more than 20 μm, preferentially 15 μm or less.

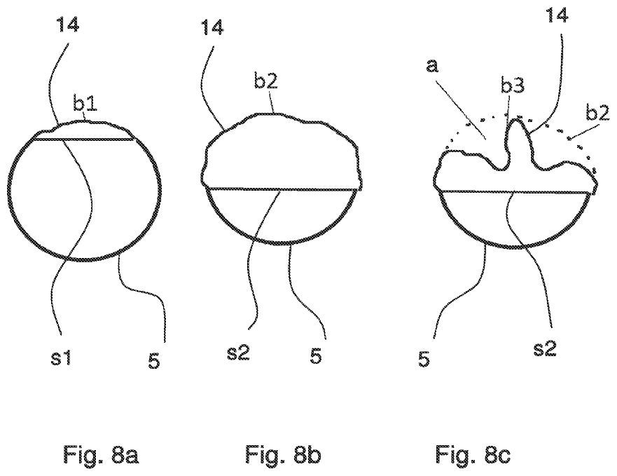

The at least one opening 9 in the tubular portion 8 exhibits, in a direction at right angles to the longitudinal extent, a circumferential-arc portion b2 covered by the membrane 14, which is substantially in alignment with the contour of the measuring tip 5 at right angles to the longitudinal extent, as can be seen in FIGS. 7 and 8b. A chord portion s2, which connects the opposing edges of the openings 9, 13, arises in a direction at right angles to the longitudinal extent of the tubular portion 8, see FIGS. 8b and 8c. In the case of a ratio of the length of the circumferential-arc portion b2 to the length of the chord s2 of more than 1, in particular between 1.33 and 1.67, preferably between 1.5 and 1.6, particularly preferably about 1.57, the membrane is able to deform particularly severely without a tensile stress arising in the membrane. Such a tensile stress would conduct some of the compressive forces from the environment into the metal tube 8 and hence falsify the result of measurement. With the configuration according to the invention and the production according to the invention, considerable scope is available for changes in the volume of the transmission liquid that are caused by changes in temperature. This becomes particularly clear from the comparison of the representations in FIGS. 8b and 8c. The length of the chord portion s2 is the same in both representations, as are the lengths of the circumferential-arc portions b2 and b3. The part a of the cross-sectional area at right angles to the longitudinal extent of the tubular portion 8 gives an indication of the change in volume of transmission liquid by virtue of a change in temperature, which in the case of a pressure-sensor device 1 according to the invention has practically no effect on the accuracy of measurement or the transmission behavior of the pressure-sensor device 1.

For comparison, FIG. 8a shows a representation of an opening 9 in the tubular portion 8 with a distinctly smaller ratio of the length of the circumferential-arc portion b1 to the length of the chord s1.

At the time of production of a pressure-sensor device 1 according to the invention, firstly at least the catheter portion 4 and the transmission liquid are expediently brought to a predetermined temperature above the operating temperature specified for the pressure-sensor device 1, preferentially about 1 K to about 5 K higher than the specified operating temperature. For the application in human beings, the predetermined temperature is preferentially about 40° C. to 46° C., in particular around 45° C. Then the lumen 7 and the measuring tip 5 are filled so far with the temperature-controlled transmission liquid, as can be discerned in FIGS. 5 and 6, that the curvature of the membrane 14 at right angles to the longitudinal extent is substantially in alignment with the contour of the measuring tip 5 at right angles to the longitudinal extent, in particular does not protrude beyond the contour of the measuring tip 5 at right angles to the longitudinal extent, as can be seen in FIG. 7. Subsequently bubble-free sealing of the lumen 7 takes place. With a following quality control, it has to be ensured that no air bubbles are trapped in the pressure-sensor device 1, because these would dramatically impair the pressure-transmission properties and render the pressure-sensor device 1 unusable.

The filling quantity of the transmission liquid has accordingly been determined in such a way that at a predetermined temperature of the transmission liquid the curvature of the membrane 14 at right angles to the longitudinal extent is substantially in alignment with the contour of the measuring tip 5 at right angles to the longitudinal extent.

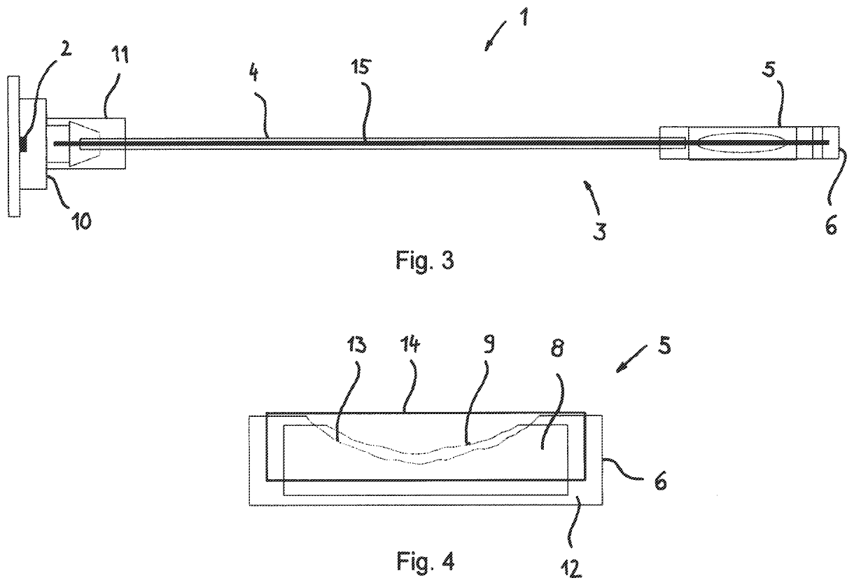

As shown in FIG. 3, in another preferred embodiment a flexible core 15 has been embedded in the lumen 7 which contains the transmission liquid. As a result, the danger of a kinking of the catheter portion 4, which might lead to a squeezing of the lumen 7 and hence to a failure of the transmission of pressure to the at least one pressure-measurement transformer 2, is reduced. Even in the event of an excessive bending of the catheter portion 4, the walls of the lumen 7 are kept spaced apart by the core 15, so that a sufficient cross-sectional area is always kept open for the transmission of pressure by the transmission liquid. By virtue of the core 15 in the lumen 7, furthermore the quantity of transmission liquid is reduced, which lessens not only the production costs for a pressure-sensor device 1 according to the invention but also the influence of thermal expansion of the transmission liquid on the accuracy of measurement.

The core 15 preferably consists of a polyamide, preferentially of a polyamide 11 or a polyamide 12. A core consisting of polyamide 12 (PA12) offers the particular advantage that a pressure-measuring device 1 according to the invention can be employed effectively also in the course of surgical interventions under ongoing X-ray control, since in such an embodiment the core 15 is resistant to X-radiation. Furthermore, a core consisting of polyamide 12 offers particular kink resistance of the catheter portion 4 of the probe 3 and hence a particularly high level of operational safety of the pressure-measuring device 1 according to the invention when employed under cramped spatial conditions, for example in an ambulance, in an air ambulance, in a mobile hospital or in a mobile medical facility. A core consisting of polyamide 11 (PA11) offers the merit of the applicability of common sterilizing procedures, is autoclavable, can be sterilized chemically with ethylene oxide, and by irradiation by means of gamma radiation. A core consisting of polyamide 11 also offers a high level of kink protection for the catheter portion 4 of the probe 3. In addition, a core consisting of a polyamide 11 is to be classified as physiologically harmless.

The invention can be realized economically particularly well with a measuring system containing at least one such pressure-sensor device. It is particularly advantageous that the catheter portion 4 of the probe 3 with the measuring tip 5 can be implanted—for example, inserted into a blood vessel—into a human or animal body via a port having only a slightly larger inner diameter. The part of the pressure-sensor device 1 containing the pressure-measurement transformer 2 with associated electronics can remain outside the body. By virtue of such an arrangement, the merits of a direct blood-pressure measurement can be combined in economically advantageous manner with the lower regulatory requirements of a passive implant. In addition, this enables an in-vivo use of the pressure-sensor device 1 also with concurrent use of minimally invasive techniques having an elevated potential for electromagnetic interference, for example, an ablation catheter.

What is claimed is:

1-14. (canceled)

15. A pressure-sensor device for a medical in-vivo application, with at least one pressure-measurement transformer and with an implantable probe, the probe being connected proximally to the at least one pressure-measurement transformer, the probe further including a catheter portion and a measuring tip at the distal end of the probe, the probe having a longitudinal extent along the catheter portion,
wherein the catheter portion exhibits at least one lumen for establishing a fluid connection from the measuring tip to the pressure-measurement transformer, and the lumen has been filled with a transmission liquid,
wherein the measuring tip includes a tubular portion with at least one laterally arranged opening, the at least one opening being covered by an elastic membrane, and the filling quantity of the transmission liquid having been determined in such a way that at a predetermined temperature of the transmission liquid the curvature of the membrane at right angles to the longitudinal extent is substantially in alignment with the contour of the measuring tip at right angles to the longitudinal extent.

16. The pressure-sensor device according to claim 15, wherein the tubular portion of the measuring tip is encased by an elastic hose, and the membrane consists of a material similar to that of the hose, preferentially of a polyurethane.

17. The pressure-sensor device according to claim 16, wherein the elastic hose exhibits an opening which overlaps the opening in the tubular portion, and the membrane has been tightly connected to the hose, the opening in the hose preferentially being slightly larger than the opening in the tubular portion.

18. The pressure-sensor device according to claim 15, wherein the tubular portion has been formed from a metal tube, preferentially from a medical stainless steel or a titanium alloy.

19. The pressure-sensor device according to claim 15, wherein the at least one opening in the tubular portion has a larger dimension in the direction of longitudinal extent than in the circumferential direction, preferentially the ratio of the dimension in the direction of longitudinal extent to the dimension in the circumferential direction amounts to at least 5:1.

20. The pressure-sensor device according to claim 15, wherein the at least one opening in the tubular portion exhibits, in a direction at right angles to the longitudinal extent, a circumferential-arc portion (b2) covered by the membrane, which is substantially in alignment with the contour of the measuring tip at right angles to the longitudinal extent, and a chord portion (s2) which connects the opposing edges of the opening, the ratio of the length of the circumferential-arc portion (b2) to the length of the chord (s2) amounting to at least 1, preferentially between 1.33 and 1.67.

21. The pressure-sensor device according to claim 15, wherein the transmission liquid comprises a water-insoluble perfluorinated liquid, the perfluorinated liquid having a boiling-point at normal pressure of at least 150° C., preferentially of about 165° C., and being completely evaporable.

22. The pressure-sensor device according to claim 15 wherein the transmission liquid has a kinematic viscosity from about 2 mm²/s to 2.2 mm²/s at 25° C. and/or a coefficient of expansion of 0.0012 K⁻¹ and/or a surface tension of about 16 mN/m.

23. The pressure-sensor device according to claim 15, wherein a flexible core has been embedded in the lumen which contains the transmission liquid.

24. The pressure-sensor device according to claim 23, wherein the core consists of a polyamide, preferentially of a polyamide 11 or a polyamide 12.

25. The pressure-sensor device according to claim 15, wherein the predetermined temperature lies above the temperature range within which the pressure-sensor device is to be employed, preferentially about 1 K to about 5 K higher.

26. The pressure-sensor device according to claim 15, wherein the predetermined temperature amounts to about 40° C. to 46° C.

27. A measuring system containing at least one pressure-sensor device according to claim 15.

28. A method for producing a pressure-sensor device for a medical in-vivo application, with an implantable probe,
wherein the probe comprises a catheter portion and a measuring tip at the distal end of the probe,
wherein the probe has a longitudinal extent along the catheter portion, the catheter portion exhibiting at least one lumen for receiving a transmission liquid,
wherein the measuring tip includes a tubular portion with at least one laterally arranged opening, the at least one opening being covered by an elastic membrane,
comprising the following steps:
controlling the temperature of the probe and of the transmission liquid to a predetermined temperature above the operating temperature specified for the pressure-sensor device, preferentially about 1 K to about 5 K higher than the specified operating temperature, to about 40° C. to 46° C.; and
filling the lumen and the measuring tip with the temperature-controlled transmission liquid so far that the curvature of the membrane at right angles to the longitudinal extent is substantially in alignment with the contour of the measuring tip at right angles to the longitudinal extent, does not protrude beyond the contour of the measuring tip at right angles to the longitudinal extent, and bubble-free sealing of the lumen.
